(19) United States
(12) Patent Application Publication
(10) Pub. No.: US 2003/0039704 A1
(43) Pub. Date: Feb. 27, 2003

(54) Dermatological preparations

(76) Inventors: Moshe Arkin, Kfar Shmaryahu (IL); Marcel Zighelboim, Kiryat-Motzkin (IL); Ilana Lavon, Lehavim (IL); Amira Zeevi, Omer (IL)

Correspondence Address: BANNER & WITCOFF, 1001 G STREET N W, SUITE 1100, WASHINGTON, DC, 20001, US

(21) Appl. No.: 10/097,434

(22) Filed: Mar. 15, 2002

(30) Foreign Application Priority Data
Mar. 15, 2001 (IL) 142,037

(51) Int. Cl. A61K 38/13; A61K 31/7048; A61K 31/65; A61K 31/415; A61K 33/04

(52) U.S. Cl. 424/705; 514/9; 514/154; 514/161; 514/28; 514/421; 514/171; 514/406; 514/569; 514/570; 514/192; 514/314

(57) ABSTRACT

The invention provides a method of treating or preventing rosacea, comprising topical administration, of a pharmaceutical preparation comprising a nonsteroidal anti-inflammatory drug (NSAID).

### FIELD OF INVENTION

The present invention relates to pharmaceutical preparations, which are very efficient in treating rosacea. More particularly, it relates to pharmaceutical preparations comprising a nonsteroidal anti-inflammatory drug (NSAID) as the therapeutic agent.

### DESCRIPTION OF THE PRIOR ART

Rosacea is a chronic inflammatory disorder, usually beginning in middle age or later, mainly affecting the face and is characterized by telangiectasia, erythema, papules and pustules¹.

The precise cause for rosacea remains unclear, but the disease is most common in persons with fair complexion, usually in their middle age or later².

Rosacea, which is sometimes called Acne Rosacea, is a distinct skin disorder, characterized by episodic flushing of affected areas, which may be associated with consumption of alcohol, hot drinks, or spicy foods. The rosacea typical redness is associated with dilation of the blood vessels in the skin, which allow more blood to flow and pool under the surface of the skin. A characteristic symptom of Rosacea is thin redlines on the face which are called telangiectasia. These redlines are the dilated blood vessels that become distended under the surface of the skin. During inflammatory episodes, affected areas of the skin, primarily the convexities of the face, develop swelling, papules, and pustules.

Histopathologic findings in rosacea dermatitis include vascular dilatation of the small vessels with perivascular infiltration of histiocytes, lymphocytes, and plasma cells. Dermal changes include loss of integrity of the superficial dermal connective tissue with edema, disruption of collagen fibers, and frequently severe elastosis.

Despite their similarities, acne vulgaris and rosacea have different pathophysiologies. The microcomedo, the primary lesion of acne, arises in response to hormonal stimuli and bacteria in people genetically predisposed to the disorder. By contrast, rosacea emerges idiopathically with no discernible inheritance patterns. The skin lesions in rosacea are notable for the absence of comedones, which distinguishes this disorder from acne vulgaris.

The differential diagnosis of rosacea also includes seborrheic dermatitis, lupus erythematosus, syphilis, tuberculosis, periorbital dermatitis, lupus malaris disseminatus, erysipelas, polymorphous light eruption, actinic reticuloid, and chronic topical corticosteroid therapy⁸.

Steroid rosacea is the name given to a rosacea-like condition on the face caused by potent topical steroids. It is a variant of perioral dermatitis (also known as periorificial dermatitis). Rosacea-like syndrome is characterized by the eruption of pinhead-sized, reddish micropapules scattered on the cheeks and forehead.

Rosacea-like syndrome is distinguished from rosacea by the absence of pustulation, keratitis and flush. Evaluation of histopathological biopsies of the papules reveals a different microscopic structure, compared to classic rosacea^{7,} which supports the conclusion that, while rosacea and rosacea-like syndrome may appear related, they are separate dermatological conditions. Rosacea-like syndrome usually resolves upon careful withdrawal from the steroid therapy. Oral tetracycline is often prescribed and may be required for several months. There was an attempt to treat rosacea-like dermatitis with the NSAIDs bufexamac and suprofen. There is no specific reference to rosacea as being the treated disease.

While there are many known treatments for acne, the only established topical treatment for rosacea comprises metronidazole gel, cream or lotion^{3,4}, which demonstrates efficacy during the inflammatory episodes of rosacea. However, metronidazole has shown evidence of carcinogenic activity in a number of studies involving its administration in mice and rats. It has also shown evidence of mutagenic activity in several in vitro bacterial assay systems⁵. Significant efforts have been made in an attempt to locate an alternative safe and efficacious therapy for rosacea, however, alternative therapies have yet to be identified.

EP 0 270 316 (A3) Patent describes topical compositions comprising 1-substituted imidazole and NSAIDs for treatment of acne.

The preferred 1-substituted imidazoles for treating acne, according to the patent, are clotrimazole, econazole, ketoconazole, miconazole and tioconazole, which are known as anti-fungal agents⁶.

These substances are characterized by a relatively large lipophilic group, attached to the ring. This group determines the level of the lipophilic nature of the specific molecule. The patent specifies that the presence of a NSAIDs together with the 1-substituted imidazole, augments the activity of the later by exhibiting a synergistic effect; e.g. the 1-substituted imidazole is, actually, the therapeutic agent that acts against acne, while the NSAID serves only as an enhancer for the therapeutic activity of the former. EP 0 270 316 (A3) does not specify that NSAID as a sole agent can be a treatment for acne and obviously not for rosacea.

NSAIDs have been used to relieve mild to moderate pain; they are also used for fever and inflammation. Such drugs are usually administered systemically and most often by oral administration. Certain NSAIDs are also available in topical dosage form. The most common side effect occurring during therapy with NSAIDs are generally gastrointestinal disturbances.

In correspondence, published by the American Medical Association, oral administration of NSAID's, such as Motrin (oral ibuprofen), in full doses (tablets of 800 mg) is suggested during periods of high activity of rosacea. However, this modality cannot be recommended due to the known gastrointestinal side effects, which occur in high incidence of 4-16%.

There remains a need therefore for a safe and effective treatment for rosacea to supplement or supplant current treatments.

### SUMMARY OF THE INVENTION

The present invention comprises pharmaceutical preparations having enhanced efficacy for topical treatment of rosacea. These preparations comprise an NSAID as the therapeutic agent for the topical treatment of rosacea.

Another aspect of this invention involves the use of an NSAID in combination with a nitroimidazole, for the treatment of rosacea.

### BRIEF DESCRIPTION OF THE DRAWING

The invention is described with reference to the following drawings, which are presented for the purpose of illustration only and are not intended to be limiting of the invention.

FIG. 1 is a plot of the number of patients having a global assessment rating of response to the tested agent of “good”, :excellent” and “completely cleared.”

### DETAILED DESCRIPTION OF THE INVENTION

It has been unexpectedly discovered that a therapeutic level of non-steroidal anti-inflammatory drugs (NSAIDs) may be administrated topically for an effective treatment or prevention of rosacea.

The use of NSAIDs in a topical preparation to treat rosacea was not described elsewhere and, hence, is novel.

Current NSAIDs are classified according to their chemical structure, as follows:

Salicylic acid derivatives (e.g., aspirin, sodium salicylate, choline magnesium trislicylate, salsalate, diflunisal, salicylsalicylic acid, sulfasalazine, olsalazine).

Para-aminophenol derivatives (e.g., acetaminophen)

Indole and indole acetic acids (e.g., indomethacin, sulindac, etodolac).

Aryl acetic acids (e.g., tolmetin, diclofenac, ketorolac)

Arylpropionic acids (e.g., ibuprofen, naproxen, flubiprofen, ketoprofen, fenoprofen, oxaprozin)

Anthranilic acids (fenamates) (e.g., mefenamic acid, meclofenamic acid)

Enolic acids (e.g., oxicams (piroxicam, tenoxicam), pyrazolidinediones (phenylbutazone, oxyphenthratrazone)

Alkanones (e.g., nabumetone).

During our explorations for new uses of NSAIDs, it was surprisingly discovered that such non-steroidal anti-inflammatory drugs, when applied topically, can alleviate the various symptoms of rosacea. The topical administration of NSAIDs has advantages over the oral route; namely, by-passing the side effect associated with this drug, and targeting the drug to the site of action. Notably, in our clinical trials, the topical NSAIDs affected simultaneously the various dermatological manifestations of rosacea. It significantly reduced the number of papules and pustules and the intensity of erythema, and telangiectasia, without causing any significant side effects. Thus, topical NSAIDs represent a new and improved therapeutic option for this multi-factorial disease. Topical administration of NSAIDs for the treatment and/or prevention of rosacea has a remarkable advantage over the treatment of choice, metronidazole, in terms of safety and avoidance of adverse effects associated with use of metronidazole.

By way of example, the NSAID is chosen from the group of piroxicam, aspirin, carprofen, diclofenac, fenoprofen, flufenamic acid, flurbiprofen, ibufenac, ibuprofen, indomethacin, isoxicam, ketoprofen, meclofenamic acid, naproxen, oxaprozin, pranoprofen, tenoxicam, zomepirac, diflunisal, sulindac and tolmetin, or combinations thereof. In at least some embodiments, the NSAID is piroxicam or tenoxicam or combinations thereof. In at least some embodiments, the NSAID is tolmetin, diclofenac, or ketorolac or combinations thereof.

In at least some embodiments, the NSAID's, for the treatment and/or prevention of rosacea, are formulated in a pharmaceutically acceptable carrier, which is suitable for use in contact with the skin, and/or which is desirably capable of dissolving or immersing the therapeutically active amounts of the NSAID. In at least some embodiments, the NSAID is in the range of about 0.1-5 wt. % of the formulation.

Additives to such compositions include, but are not limited to, water, surfactants, emulsifiers, diglycerides, triglycerides, stabilizing agents, thickening agents, alpha-hydroxy carboxylic acids, antioxidants, preservatives, moisturizers, petroleum, mineral oil, glycerol, ethanol, propanol, isopropanol, butanol, polymeric gelling agents, flavoring, colorant and odorant agents and other formulation components, used in the art of pharmaceutical and cosmetic formulary. It would be apparent to those of ordinary skill in the art of dermatology that the resulting compositions can be in many forms including, but not limited to, liquids, solutions, lotions, creams, pastes, emulsions, gels, soap bars, sprays or aerosols. Such compositions may be applied manually, or using various application devices.

The composition for the treatment and/or prevention of rosacea may consist of a single NSAID or a combination of more than one NSAIDs. The composition may further contain additional therapeutic agents, which can add to the efficacy of treatment. Examples of such therapeutic agents are antibiotic, antibacterial, antiviral, anesthetic, analgesic, antiallergic, retinoid, anti-histamine, sulfur, immunosuppressant and antiproliferative medications, and mixtures thereof in any proportion. The concentration of said therapeutic agents may be adopted to exert a therapeutic effect on a disease when applied to an afflicted area. In at least some embodiments, the additional therapeutic agent is a nitro-imidazole, such as metromidazole, and the additional therapeutic agent is present in the range of about 0.1-5% w/w.

In at least some embodiments, a class of therapeutic agents, which can accompany the NSAID, comprises antibacterial agents. Such agents can contribute to the therapeutic affect by affecting the bacterial aspect of the disease, as well as alleviating any secondary infection. The antibacterial drug can be active against gram positive and gram negative bacteria, aerobic bacteria and anaerobic ones.

In at least some embodiments, the antibacterial agent is a nitro-imidazole, such as metronidazole, which is, by itself, effective in the treatment of rosacea. Hence, use of a mixture containing a NSAID and metronidazole is especially preferred since it is believed that such a mixture affords a synergistic effect.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples serve only to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLE 1

Piroxicam Gel formulation

| Ingredient | % w/w |
|---|---|
| Piroxicam | 0.5 |
| Ethanol 95% | 18 |
| Triethanolamine | 0.6 |
| Diethyleneglycol monoethyl ether | 17 |
| (Transcutol ®) | |
| Methylparaben | 0.027 |
| Propylparaben | 0.014 |
| Brij 35 | 0.6 |
| Arlacel 186 | 0.016 |
| Cetiol HE | 6 |
| Hydroxyethyl cellulose | 1.4 |
| Purified water | q.s |

Procedure:

1. In stainless steel vessel Cetiol HE and Arlacel 186 were mixed until dissolves.

2. In another stainless steel vessel purified water and Brij 35 were mixed and homogenized for 30 minutes until dissolves.

3. The following ingerdients were added stepwise to the mixer, in the following order:

i. Triethanolamine and Transcutol

ii: Piroxicam, Methylparaben and Propylparaben,

iii. Ethanol 95%,

iv. Mixture from step #1

v. Mixture from step #2,

4. Hydroxyethyl Cellulose added and the mixture washomogenized for 30 minutes

5. The content of the mixer was further mixed for 1 hour.

The above composition was further used in clinical trials, as exemplified below.

### EXAMPLE 2

Piroxicam and Metronidazole Gel Formulation

| Ingredient | % w/w |
|---|---|
| Piroxicam | 0.5 |
| Metronidazole | 0.75 |
| Ethanol 95% | 18 |
| Triethanolamine | 0.6 |
| Diethyleneglycol monoethyl ether | 17 |
| (Transcutol ®) | |
| Methylparaben | 0.027 |
| Propylparaben | 0.014 |
| Brij 35 | 0.6 |
| Arlacel 186 | 0.016 |
| Cetiol HE | 6 |
| Hydroxyethyl cellulose | 1.4 |
| Purified water | q.s. |

The formulation was essentially produced according to the procedure, described in Example 1.

### EXAMPLE 3

Diclofenac Gel Formulation

| Ingredient | % w/w |
|---|---|
| Diclofenac | 1 |
| Ethanol 95% | 18 |
| Triethanolamine | 0.6 |
| Diethyleneglycol monoethyl ether | 17 |
| (Transcutol ®) | |
| Methylparaben | 0.027 |
| Propylparaben | 0.014 |
| Brij 35 | 0.6 |
| Arlacel 186 | 0.016 |
| Cetiol HE | 6 |
| Hydroxyethyl cellulose | 1.4 |
| Purified water | q.s. |

The formulation was essentially produced according to the procedure, described in Example 1.

### EXAMPLE 4

Diclofenac and Metronidazole Gel Formulation

| Ingredient | % w/w |
|---|---|
| Diclofenac | 1 |
| Metronidazole | 0.75 |
| Ethanol 95% | 18 |
| Triethanolamine | 0.6 |
| Diethyleneglycol monoethyl ether | 17 |
| (Transcutol ®) | |
| Methylparaben | 0.027 |
| Propylparaben | 0.014 |
| Brij 35 | 0.6 |
| Arlacel 186 | 0.016 |
| Cetiol HE | 6 |
| Hydroxyethyl cellulose | 1.4 |
| Purified water | q.s. |

The formulation was essentially produced according to the procedure, described in Example 1.

### EXAMPLE 5

Piroxicam and Metronidazole Cream Formulation

| Ingredient | % w/w |
|---|---|
| Metronidazole | 0.75 |
| Piroxicam | 0.5 |
| Emulsifying Wax | 12.2 |
| Sorbitol 70% solution | 4 |
| Glycerin | 4.1 |
| Isopropyl Palmitate | 2 |
| Benzyl Alcohol | 1 |
| Lactic Acid | q.s |
| Sodium Hydroxide | q.s |
| Purified Water | q.s |

### EXAMPLE 6

Piroxicam and Acyclovir Gel Formulation

| Ingredient | % w/w |
|---|---|
| Piroxicam | 0.5 |
| Acyclovir | 5 |
| Carbopol | 1 |
| Oleic Acid | 3 |
| Propylene Glycol | 38 |
| Sodium Hydroxide | q.s |
| Purified Water | q.s |

### EXAMPLE 7

Clinical Trial I

This study was randomized and double blind active-drug-controlled study. Only in case of emergencies, the investigators had access to the randomization code.

A total of 40 patients with moderate to severe rosacea were enrolled.

Patients were assigned consecutive patient study numbers (No. 1-No. 40) according to the chronological entry into the study. The treatment each patient received was assigned through randomization.

Each patient applied topically Metronidazole 0.75% Gel to one side of the face and Piroxicam 0.5% Gel prepared as described in Example 1 to the other side of the face. Both drugs had similar appearance. They were supplied in identical tubes, bearing “Left” or “Right” labels, according to randomization list. Neither the Investigator, nor the patients knew the identity of any of the supplied drugs and thus, complete blindness was maintained.

Application was performed twice daily for 15 weeks. Patients were examined at baseline (treatment initiation) and after 3, 6, 9 and 15 weeks of treatment. At each visit the investigator counted the papules, pustules, telangiectasia, global assessment of disease severity and adverse effects. 30 patients out of 40 were eligible for efficacy evaluation at Week 15.

Patient were enrolled according to the following criteria:

Patients of both sexes aged 21-70 years

Clinically proven bilateral stable acne rosacea

Rosacea grading moderate to severe.

Total facial papules and pustules >10 at both sides.

Written informed consent must be obtained and documented in the patient record.

### EFFICACY RESULTS

1. Baseline Comparison of the Papules, Pustules and Total Lesion Counts

The mean baseline total lesion count in each treatment side was in the range of 8.08-8.35 and the minimum total lesion count was 4 in each side of the face (total of at least 8 in two sides of the face).

The number of papules and pustules and the mean total lesion counts of both treatment sides at baseline and after 3, 6, 9 and 15 weeks of treatment are summarized in Table 1, demonstrating that both drugs are efficacious in treatment of papules and pustules. There was a statistically significant difference in the number of papules, pustules and total lesion counts for both drugs between baseline and each of the following visits (in all cases, p<0.0001).

TABLE 1
Number of Lesions at Baseline and after 3, 6, 9 and 15 Weeks of
Treatment - Papules, Pustules and Total Lesion Count (Papules + Pustules)

| | | Visit 1 | Visit 2 | Visit 3 | Visit 4 | Visit 5 |
|---|---|---|---|---|---|---|
| | | (Baseline) | (Week 3) | (Week 6) | (Week 9) | (Week 15) |
| Piroxicam 0.5% | Mean Papules | 6.23 | 2.67 | 1.83 | 1.43 | 1.53 |
| Gel | Mean Pustules | 1.45 | 0.46 | 0.25 | 0.18 | 0.33 |
| | Mean Total | 8.08 | 3.13 | 2.08 | 1.60 | 1.87 |
| Metronidazole | Mean Papules | 6.90 | 2.77 | 2.08 | 1.54 | 1.77 |
| 0.75% | Mean Pustules | 1.45 | 0.56 | 0.28 | 0.29 | 0.37 |
| | Mean Total | 8.35 | 3.33 | 2.36 | 1.83 | 2.13 |

TABLE 2
Percent Change from Baseline - Papules, Pustules and Total
Lesion Count (Papules + Pustules)

| | | Visit 2 | Visit 3 | Visit 4 | Visit 5 |
|---|---|---|---|---|---|
| | | (Week | (Week | (Week | (Week |
| | | 3) | 6) | 9) | 15) |
| Piroxicam 0.5% | Mean Papules | 56 | 71 | 79 | 78 |
| Gel | Mean Pustules | 66 | 90 | 92 | 85 |
| | Mean Total | 59 | 75 | 82 | 80 |
| Metronidazole | Mean Papules | 59 | 71 | 78 | 77 |
| 0.75% | Mean Pustules | 73 | 89 | 79 | 88 |
| | Mean Total | 59 | 71 | 78 | 77 |

As shown in Tables 1 and 2, both Piroxicam 0.5% and Metronidazole 0.75% treatments resulted in clinically relevant and statistically significant reduction of papules and pustules after 3 weeks. 56-59% improvement in papules and 66-73% improvement in pustules. This effect was more pronounced after 6 weeks of treatment and reached its maximum after 9 weeks of treatment 78-79% improvement in papules and 79-92% improvement in pustules. In all cases, p-value was less than 0.0001.

2. Erythema

Erythema scoring was assessed at all visits and was graded as follows: 0=none, 1=mild, 2=moderate, 3=severe.

Table 3 demonstrates that both treatments were effective in reducing erythema starting from visit 2. It can also be seen that both treatments are equally efficacious in treating erythema.

TABLE 3
Erythema Values at Visits 1-5

| | | Visit 1 | Visit 2 | Visit 3 | Visit 4 | Visit 5 |
|---|---|---|---|---|---|---|
| | | (Baseline) | (Week 3) | (Week 6) | (Week 9) | (Week 15) |
| Piroxicam 0.5% | Mean | 2.65 | 2.03 | 1.9 | 1.83 | 2.1 |
| Gel | Median | 3 | 2 | 2 | 2 | 2 |
| p-Value (vs. Baseline) | | | 0.0001 | 0.0001 | 0.0001 | 0.0003 |
| Metronidazole | Mean | 2.63 | 2.00 | 1.86 | 2.00 | 2.07 |
| 0.75% | Median | 3 | 2 | 2 | 2 | 2 |

| | | | | |
|---|---|---|---|---|
| p-Value (vs. Baseline) | 0.0001 | 0.0001 | 0.0001 | 0.0001 |

3. Telangiectasia

Telangientasia were measured at baseline and in all other visits and the values at each visit are presented in Table 4. As shown in Table 6 There was a statistically significant difference from baseline values of telangiectasia for Piroxicam in Visits 2-5 and for Metronidazole, in Visits 2 and 3. It should be noted that other topical medications for rosacea, including those containing metronidazole are not affective in treating telangiectacia.

TABLE 4
Number of Telangiectasia Lesions at Visits 1-5

| | | Visit 1 | Visit 2 | Visit 3 | Visit 4 | Visit 5 |
|---|---|---|---|---|---|---|
| | | (Baseline) | (Week 3) | (Week 6) | (Week 9) | (Week 15) |
| Piroxicam 0.5% | Mean | 5.00 | 4.49 | 4.36 | 4.03 | 3.70 |
| Gel | Median | 4 | 4 | 4 | 4 | 4 |
| p-Value (vs. Baseline) | | | 0.0376 | 0.0354 | 0.0082 | 0.0087 |
| Metronidazole | Mean | 4.83 | 4.33 | 4.08 | 4.00 | 3.83 |
| 0.75% | Median | 5 | 4 | 4 | 3 | 4 |

| | | | | |
|---|---|---|---|---|
| p-Value (vs. Baseline) | 0.0194 | 0.046 | 0.0853 | 0.0704 |

4. Responder's Rate

The above data was further processed, to assess rate of response, when “response” was defined as either more than 50% improvement or more than 75% improvement in each of the quantitative efficacy parameters.

As shown in Table 5, the portion of Piroxicam-treated sites with 75% was higher than the respective portion of Metronidazole-treated sites, indicating that Piroxicam is slightly more efficacious than Metronidazole.

TABLE 5
Rate of Response when “Response” was Defined as either >50% or
75% improvement

| | | Visit 2 | | Visit 3 | | Visit 4 | | Visit 5 | |
|---|---|---|---|---|---|---|---|---|---|
| | | (Week 3) | | (Week 6) | | (Week 9) | | (Week 15) | |
| Number of Subjects | | 39 | | 36 | | 35 | | 30 | |
| % Improvement | | >50% | >75% | >50% | >75% | >50% | >75% | >50% | >75% |
| Piroxicam | Papules | 77 | 21 | 86 | 58 | 91 | 71 | 83 | 70 |
| 0.5% Gel | Pustules | 77 | 64 | 97 | 83 | 94 | 86 | 90 | 90 |
| | Total Lesions | 79 | 26 | 92 | 67 | 91 | 74 | 87 | 73 |
| | Telangiectasia | 10 | 5 | 19 | 6 | 31 | 11 | 40 | 23 |
| | Erythema | 15 | 0 | 28 | 0 | 31 | 0 | 27 | 0 |
| Metronidazole | Papules | 82 | 15 | 89 | 50 | 91 | 74 | 87 | 60 |
| 0.75% | Pustules | 79 | 67 | 89 | 83 | 91 | 83 | 90 | 87 |
| | Total Lesions | 85 | 21 | 89 | 56 | 91 | 74 | 87 | 67 |
| | Telangiectasia | 10 | 5 | 19 | 6 | 23 | 11 | 30 | 23 |
| | Erythema | 21 | 0 | 28 | 0 | 20 | 0 | 27 | 0 |

5. Global Assessment of Improvement vs. Baseline

The Investigator's global assessment of treatment success rate was defined as the percentage of patients with greater than good response to treatment at each visit, compared to baseline.

The magnitude of response was be assessed as follows:

complete clearing (no sign or symptom of disease)

excellent response: 75-99% improvement

good response: 50-74% improvement

fair response: ? 50%

poor response: no detectable improvement

condition worsened.

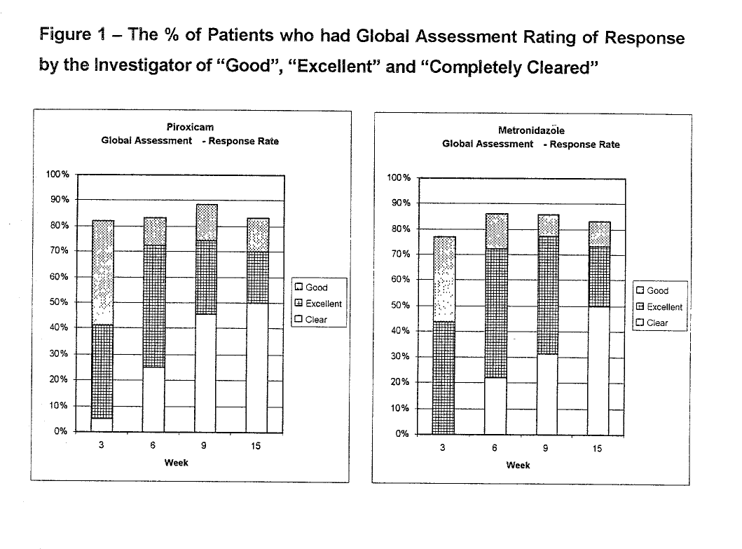

Table 6 summarizes the global assessment and FIG. 1 illustrates the me results graphically. It is noted that even after 3 weeks of treatment (Visit 2), the total number of patients who were rated as demonstrating more than good response, exceeded 80% of all patients for both Piroxicam and Metronidazole. Yet, complete clearance was more rapid in the Piroxicam-treated sites.

TABLE 6
Subjective Assessment of Improvement vs. Baseline by the
Investigator

| | | Visit 2 | | Visit 3 | | Visit 4 | | Visit 5 | |
|---|---|---|---|---|---|---|---|---|---|
| | | n | % | n | % | n | % | n | % |
| Piroxicam | 1 - Clear | 2 | 5 | 9 | 25 | 16 | 46 | 15 | 50 |
| 0.5% Gel | 2- Excellent | 14 | 36 | 17 | 47 | 10 | 29 | 6 | 20 |
| | 3- Good | 16 | 41 | 4 | 11 | 5 | 14 | 4 | 13 |
| | 1 + 2 + 3 | 32 | 82 | 30 | 83 | 31 | 89 | 25 | 83 |
| Metronidazole | 1 - Clear | 0 | 0 | 8 | 22 | 11 | 31 | 15 | 50 |
| 0.75% | 2- Excellent | 17 | 44 | 18 | 50 | 16 | 46 | 7 | 23 |
| | 3- Good | 13 | 33 | 5 | 14 | 3 | 9 | 3 | 10 |
| | 1 + 2 + 3 | 30 | 77 | 31 | 86 | 30 | 86 | 25 | 83 |

Although no statistically significant difference between the compared drugs was found In the main efficacy parameters, the physicians global assessment of the two treatments yielded a marked difference between the drugs. The use of Piroxicam 0.5% gel was involved with less adverse events reported by the patients.

Piroxicam gel exhibits a clear trend to more efficacious and safer than the Metronidazole gel. Due to the ability of this NSAID preparation to alleviate each of the symptoms of rosacea, it is believed that it is advantageous for both treating and preventing the reccurence of this dermatological disorder.

As it has been mentioned before, Metronidazole has shown evidence of carcinogenic and mutagenic activity. This study demonstrates the efficacy of Piroxicam 0.5% gel in the treatment of rosacea. As being a safer therapeutic agent in terms of adverse effects involved in its use, Piroxicam is the preferred treatment for rosacea,

### Example 8

Clinical Trial II

The efficacy of two topical NSAID, Piroxicam and Diclofenac, was evaluated in a prospective comparative study, in the treatment of patients with mild to moderate facial Rosacea. Each patient had one side of his face treated with Piroxicam gel and the other side treated with Diclofenac gel. After 6 weeks of study, 70% of the patients showed improvement for both drugs.

The results of this study show that the NSAID's anti-rosacea effect is general can be attained with various NSAIDs.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

References:

¹ The Merck Manual of Diagnosis and Therapy, 6^{th} Ed., p. 2432, 1992.

² Martindale 31^{st} Ed., p. 1080.

³ Bitar A, et al. A double-blind randomised study of metronidazole (Flagyl) 1% cream in the treatment of acne rosacea: a placebo-controlled study, Drug Inves 1990; 2:242-8.

⁴ Schmadel LK et al, Topical metronidazole: a new therapy for rosacea, Clin. Pharm. 1990; 9:94-101.

⁵ PDR 2000 electronic library

⁶ Martindale 31^{st} Ed., p. 393.

⁷ Textbook of Dermatology, Arthur Rook, 1615-1617

⁸ Leyden, et al. Am.Steroid rosacea Arch Dermatol. 1974;1 10:619.

What is claimed is:

1. A method of treating or preventing rosacea, comprising topical administration, of a pharmaceutical preparation comprising a nonsteroidal anti-inflammatory drug (NSAID).

2. A method, according to claim 1, wherein the NSAID is selected from the group consisting of salicylic acid derivatives, indole and indende acetic acids, heteraryl acetic acid derivatives, arylpropionic acids, anthranilic acids (fenamates), enolic acids, pyrazolidinediones and alkanones and combinations thereof.

3. A method, according to claim 1, wherein the NSAID is selected from the group consisting of piroxicam, aspirin, carprofen, diclofenac, fenoprofen, flufenamic acid, flurbiprofen, ibufenac, ibuprofen, indomethacin, isoxicam, ketoprofen, meclofenamic acid, naproxen, oxaprozin, pranoprofen, tenoxicam, zomepirac, diflunisal, sulindac and tolmetin and combinations thereof.

4. The method according to claims 1, 2 or 3, wherein the NSAIDs in present in an amount in the range of about 0.1-5% w/w

5. A method according to claim 1, wherein the a pharmaceutical preparation further comprises a secondary therapeutic agent selected from the group consisting of antibiotic, antibacterial, antifungal, antiviral, anesthetic, analgesic, antiallergic, corticosteroid, retinoid, anti-histamine, sulfur, immunosuppressant and antiproliferative agents, and mixtures thereof.

6. A method according to claim 1, wherein the a pharmaceutical preparation further comprises an antibiotic or antibacterial agent selected from the group consisting of chloramphenicol, tetracyclines, synthetic and semi-synthesic penicillins, beta-lactames, quinolones, fluoroquinolnes, macrolide antibiotics, peptide antibiotics, cyclosporines and any combinations thereof at a therapeutically effective concentration.

7. A method according to claim 1, wherein the pharmaceutical preparation further comprises a therapeutic amount of a nitro-imidazole.

8. A method according to claim 1, wherein the a pharmaceutical preparation further comprises metronidazole.

9. A method according to claim 1, wherein the pharmaceutical preparation further comprises metronidazole in a concentration in the range of about of 0.1-5% w/w.

10. A method according to claim 1, wherein the a pharmaceutical preparation is presented in the form selected from the group consisting of a gel, a cream, an ointment, a solution, a lotion and an aerosol.

11. Use of an NSAID for the manufacture of a pharmaceutical composition for the treatment of rosacea.

12. Use of an NSAID according to claim 11, selected from the group consisting of: salicylic acid derivatives, indole and indende acetic acids, heteraryl acetic acid derivatives, arylpropionic acids, anthranilic acids (fenamates), enolic acids, pyrazolidinediones and alkanones and combinations thereof, for the manufacture of a pharmaceutical composition for the treatment of rosacea.

13. Use of an NSAID according to claim 11, selected from the group consisting of: piroxicam, aspirin, carprofen, diclofenac, fenoprofen, flufenamic acid, flurbiprofen, ibufenac, ibuprofen, indomethacin, isoxicam, ketoprofen, meclofenamic acid, naproxen, oxaprozin, pranoprofen, tenoxicam, zomepirac, diflunisal, sulindac and tolmetin and combinations thereof, for the manufacture of a pharmaceutical composition for the treatment of rosacea.

14. Use of an NSAID according to claim 11, together with a secondary therapeutic agent, selected from the group of antibiotic, antibacterial, antifungal, antiviral, anesthetic, analgesic, antiallergic, corticosteroid, retinoid, anti-histamine, sulfur, immunosuppressant and antiproliferative medications, and mixtures thereof, for the manufacture of a pharmaceutical composition for the treatment of rosacea.

15. A topical pharmaceutical preparation, comprising a therapeutic amount of a non-steroidal anti-inflammatory drug and a secondary therapeutic agent, selected from the group of antibiotic, antibacterial, antiviral, anesthetic, analgesic, antiallergic, corticosteroid, retinoid, anti-histamine, immunosuppressant and antiproliferative medications, and mixtures thereof.

16. A topical pharmaceutical preparation, according to claim 15, wherein the secondary therapeutic agent comprises an antibiotic or antibacterial agent selected from the group of chloramphenicol, tetracyclines, synthetic and semi-synthesic penicillins, beta-lactames, quinolones, fluoroquinolnes, macrolide antibiotics, peptide antibiotics, cyclosporines and any combination thereof at a therapeutically effective concentration.

17. A topical pharmaceutical preparation, of claim 15, wherein the secondary therapeutic agent comprises a therapeutic amount of a nitro-imidazole.

18. A topical pharmaceutical preparation, according to claim 17, wherein the nitro-imidazole is metronidazole.

19. A topical pharmaceutical preparation, according to claim 18, wherein the concentration of Metronidazole is in the range of 0.1-5% w/w.

20. A topical pharmaceutical preparation for the treatment of rosacea, comprising piroxicam.

21. A topical pharmaceutical preparation, according to claim 20, also comprising an additional therapeutic agent.

22. A topical pharmaceutical preparation, according to any of claims 15-21, presented in the form selected from the group consisting of a gel, a cream, an ointment, a solution, a lotion and an aerosol.

23. A method of treating or preventing rosacea, comprising topical administration, of a pharmaceutical preparation comprising piroxicam and optionally a secondary therapeutic agent.

24. Use of a topical pharmaceutical preparation as defined in any one of the claims 15-19 for the treatment of rosacea.
